(19) United States
(12) Patent Application Publication
(10) Pub. No.: US 2007/0167349 A1
(43) Pub. Date: Jul. 19, 2007

(54) Estrogen cancer therapy

(75) Inventor: Jack Singer, Seattle, WA (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Cell Therapeutics, Inc., Seattle, WA (US)

(21) Appl. No.: 11/634,356

(22) Filed: Dec. 5, 2006

Related U.S. Application Data
(60) Provisional application No. 60/742,725, filed on Dec. 6, 2005.
(60) Provisional application No. 60/814,221, filed on Jun. 16, 2006.

(51) Int. Cl. A61K 38/16

(52) U.S. Cl. 514/2

(57) ABSTRACT

Disclosed are methods for selecting a treatment for, and then treating various types of cancers in males and females, comprising delivery of an anti-cancer drug conjugated to a poly (amino acid) polymer, optionally in combination with estrogen therapy.

### CROSS-REFERENCE TO RELATED APPLICATIONS

This application claims the benefit of the filing date of U.S. Provisional Application No. 60/742,725, filed Dec. 6, 2005 and U.S. Provisional Application No. 60/814,221, filed Jun. 16, 2006 the disclosures of which are incorporated by reference herein.

### BACKGROUND OF THE INVENTION

Water-soluble paclitaxel conjugates, methods of making them and methods of using them to treat taxane-responsive diseases, are described in U.S. Pat. Nos. 5,977,163 and 6,262,107. As reported therein, compositions containing the conjugated forms of the drugs were found to be surprisingly effective as anti-tumor agents against exemplary tumor models, and were expected to be at least as effective as paclitaxel or docetaxel against any of the diseases or conditions for which taxanes or taxoids are known to be effective. They also provided advantages in terms of ease of formulation (by overcoming the drawbacks associated with the insolubility of the drugs themselves), controlled release, and fewer side effects (due at least in part to elimination of the need for solvents that are associated with side effects observed with prior paclitaxel compositions).

### SUMMARY OF THE INVENTION

A first aspect of the present invention is directed to a method of treating patients identified as having a premenopausal estrogen level, and who are diagnosed with cancer, comprising delivering to a female in need thereof, a conjugate comprising a poly(amino acid) polymer conjugated to an anti-cancer drug (herein referred to as the conjugated drug or drug conjugate), wherein the cancer is characterized by the presence of estrogen receptor-bearing cancer cells or estrogen receptor-bearing normal (non-diseased) cells of the organ or tissue in which the cancer originates.

A second aspect of the present invention is directed to a method of treating patients identified as having a postmenopausal estrogen level, and who are diagnosed with cancer, wherein the cancer is characterized by the presence of estrogen receptor-bearing cancer cells or estrogen receptor-bearing normal (non-diseased) cells of the organ or tissue in which the cancer originates, and wherein said treatment comprises delivering to the patient the conjugated drug and estrogen therapy.

In various embodiments of the present invention, the poly(amino acid) polymer is poly(glutamic acid) or poly(lysine); the anti-cancer drug is a taxane; in further embodiments, the anti-cancer drug is paclitaxel. In another embodiment, the anti-cancer drug is paclitaxel and the polymer comprises poly(glutamic acid). In another embodiment, the estrogen therapy comprises administration of 17-β-estradiol. In another embodiment, the cancer is lung cancer, in a further embodiment, the cancer is non-small cell lung cancer.

A further aspect of the present invention is directed to a method for selecting a cancer treatment regimen based on blood serum estrogen levels.

While not intending to be bound by any particular theory of operation, Applicant believes that the presence of estrogen (i.e., endogenous or exogenously supplied estrogen therapy) enhances the efficacy of the conjugated drugs by increasing the amount of drug delivered to the cancerous tissue (e.g., tumor). Estrogen may also cause upregulation of cathespin B, an enzyme associated with the separation of active drug from the backbone of the polymeric carrier, resulting in increased amounts of unconjugated (or free) anti-cancer drug in the cancerous tissue.

### BRIEF DESCRIPTION OF THE DRAWINGS

FIG. 1 is a line graph indicating survival, in days, for women with postmenopausal (i.e., low) blood plasma estrogen levels treated with either conjugated paclitaxel+carboplatin (CT-2103+Carbo (i.e., carboplatin); solid line with open circles) or unconjugated paclitaxel+carboplatin (Paclitaxel+Carbo (i.e., carboplatin); dashed line with open squares).

FIG. 2 is a line graph indicating survival, in days, for women with premenopausal (i.e., high) blood plasma estrogen levels treated with either conjugated paclitaxel+carboplatin (CT-2103+Carbo; solid line with open circles) or unconjugated paclitaxel+carboplatin (Paclitaxel+Carbo; dashed line with open squares).

FIG. 3 is a line graph depicting overall survival (OS) in women under age 55, treated with paclitaxel poliglumex (PPX) versus control.

FIG. 4 is a line graph depicting OS in women over age 55, treated with PPX versus control.

FIG. 5 is a line graph depicting OS in premenopausal women, treated with PPX versus control.

FIG. 6 is a western blot depicting Erα and ERβ expression in human tumor cell lines and the HT-29 and H460 tumor models.

FIG. 7 is a set of line graphs depicting the effect of estrogen on tumor weight and cathepsin B activity in the HT-29 tumor model.

FIG. 8 is a set of graphs depicting the effect of estrogen on tumor weight and cathepsin B activity in the H460 tumor model.

FIG. 9 is a western blot depicting the effect of estradiol on estrogen receptor β (ERβ, expression in the HT-29 tumor model.

FIG. 10 is a western blot depicting the effect of estradiol on ERβ expression in the H460 tumor model.

FIG. 11 is a chart depicting an RT-PCR analysis on the E-Cadherin downstream gene of the estrogen receptor signaling pathway.

FIG. 12 is a chart depicting an RT-PCR analysis on the downstream gene (Id-2) of the estrogen receptor signaling pathway.

FIG. 13 is a set of line graphs indicating the levels of unconjugated or total paclictaxel, in ng of drug/g of tissue, in either liver, lung or bone marrow of male (A), female (B) or oophorectomized female rats (C); wherein:
(A) (black solid line with open squares);
(B) (blue solid line with open squares); and
(C) (red solid line with open squares).

### DETAILED DESCRIPTION

The present invention is directed to a cancer treatment. As used herein, the term “estrogen receptor bearing cancer” refers to any cancer, tumor-forming or otherwise (e.g., hematopoietic cancers such as leukemia), characterized by the presence of cancer cells bearing estrogen receptors, or a cancer that originates in an organ or tissue containing normal (non-diseased) cells that bear estrogen receptors. The estrogen receptor may be transiently expressed and/or the estrogen receptor bearing cells may represent a fraction of the total population of cells (cancer cells or non-diseased cells). Stated somewhat differently, not all the cells associated with a particular cancer, or the cells in the originating tissue, necessarily have estrogen receptors. Nor do the cancer cells always express estrogen receptors over the course of disease progression. Nor do the normal cells in the originating tissue necessarily express estrogen receptors over their entire lifetime).

There are two forms of estrogen receptors, namely α- and β-receptors. The prevalence of either receptor on cancer cells may be related to the particular type of cancer and/or a particular stage of progression of the cancer. Thus, the cancer cells may express each form at different times during progression of the disease. See Leav et al., Am. J. Path. 159:79-92 (2001). Estrogen receptor β-bearing cancers include, but are not limited to, non-small cell lung cancer (NSCLC), breast cancer, ovarian cancer, uterine epithelial cancer and sarcomas, colon cancer, glioma, prostate cancer, testicular cancers, and melanoma. For this application, estrogen β receptors are of particular interest as they are the only functional receptors expressed in lung tissues and in NSCLC. See Patrone et al., Mol. Cell. Bio. 23:8542-8552 (2003).

As described herein, depending on the selected patient population, the methods of the invention may also comprise administration of estrogen therapy. In some embodiments of the invention, the selected patient population constitutes females with premenopausal (or high) levels of endogenous estrogen. Females with “premenopausal levels of endogenous estrogen,” as the phrase is used herein, typically have a serum or blood plasma estrogen level (typically measured in terms of the bioactive hormone estradiol (estradiol-17β, or E2, utilizing, e.g., commercially available enzyme-linked immunsorbent assay (ELISA) kits such as those from the RDI division of Fitzgerald Industries International (Concord, MA)) from about 30 to about 1000 picograms/milliliter (pg/ml), e.g., about 30 to about 300 pg/ml of serum or plasma. Premenopausal levels of endogenous estrogen are typically found in females who are post-pubescent, have. not undergone menopause, and have no other mitigating health factors that would dramatically change estrogen levels. This population typically includes females aged from 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, to about 54, and any subset range therein. In addition, although rare, males sometimes have blood serum or plasma estrogen levels similar to premenopausal females. Therefore, the term “premenopausal” as used herein, in connection with blood serum or plasma estrogen levels, also embraces males.

In other embodiments of the invention, the selected patient population constitutes patients with low levels of estrogen, generally males and postmenopausal females. In these embodiments, the patients are treated with combined therapy that entails delivery or administration of the drug conjugate and estrogen therapy. These patients typically have serum or blood plasma estrogen levels less than the lower limits of quantitation, generally less than 30 pg/ml, e.g., about 20 to just under 30 pg/ml. While it is recognized that males do not experience “menopause”, as used herein, in connection with blood serum or plasma estrogen levels, the term “postmenopausal” also embraces males. Postmenopausal females are typically over age 55. In some individual cases, however, women younger than age 55 may have postmenopausal estrogen levels. These cases include women who have had surgical oophorectomies, or lost ovarian function due to administration of cytotoxic chemotherapy for cancer or other drugs that affect ovarian or pituitary function, or have primary ovarian failure. This group may include younger females aged 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54.

Polymers useful in the present invention comprise amino acids. Examples of such polymers are described in U.S. Pat. Nos. 5,977,163 and 6,262,107. The term “poly (amino acid) polymer”, as used herein, refers to copolymers and homopolymers comprised of naturally occurring or synthetic amino acids. The amino acids need not be polymerized through peptide bonds but may be bound in any fashion that allows amino acid monomers to be bound sequentially.

Polymers that may be useful in the present invention include but are not limited to poly(l-glutamic acid), poly(d-glutamic acid), poly(dl-glutamic acid), poly(l-aspartic acid), poly(d-aspartic acid), poly(dl-aspartic acid), poly(l-lysine), poly(d-lysine), poly(dl-lysine), poly(l-serine), poly(d-serine), poly(dl-serine), poly(l-glycine), poly(d-glycine), poly(dl-glycine), poly(l-alanine), poly(d-alanine), poly(dl-alanine), poly(l-tyrosine), poly(d-tyrosine), poly(dl-tyrosine), poly(l-threonine), poly(d-threonine), poly(dl-threonine), poly(d-cysteine), poly(l-cysteine), and poly(dl-cysteine). In other embodiments, the polymers are copolymers, such as block, graft or random copolymers, of the above listed poly(amino acids) with non-amino acid polymers such as polyethylene glycol, polycaprolactone, polyglycolic acid and polylactic acid, as well as poly(2-hydroxyethyl l-glutamine), chitosan, carboxymethyl dextran, hyaluronic acid, human serum albumin and alginic acid. Poly-glutamic acid(s) and poly-lysine(s) are particularly preferred. Reference to any poly(amino acid), e.g., poly(glutamic acid) is not limiting with respect to a isomerism, and thus embraces all isomeric forms, including d, l and dl forms.

In certain embodiments, “a water soluble polyamino acid,” “water soluble polyamino acids” or “water soluble polymer of amino acids” are used. By these terms, it is meant that the polymers include amino acid chains comprising combinations of glutamic acid and/or aspartic acid and/or lysine, of either d and/or 1 isomer conformation. In certain embodiments, such a “water soluble polyamino acid” contains one or more glutamic acid, aspartic acid, and/or lysine residues. In preferred embodiments, the polymer is a poly(anionic amino acid) polymer. Examples of poly(anionic amino acid) polymers include poly-glutamic acid, poly-aspartic acid, and other homo- or hetero-amino acid polymers having a net negative charge at pH 7.

In other embodiments, the polymers are copolymers such as block, graft or random copolymers, containing glutamic acid. Thus, copolymers of glutamic acid with at least one other (preferably biodegradable) monomer, oligomer or polymer are included. These include, for example, copolymers containing at least one other amino acid, such as aspartic acid, serine, tyrosine, glycine, ethylene glycol, ethylene oxide, (or an oligomer or polymer of any of these) or polyvinyl alcohol. Glutamic acid residues may carry one or more substituents and the polymers include those in which a proportion or all of the glutamic acid monomers are substituted. Substituent groups include, for example, alkyl, hydroxy alkyl, aryl and arylalkyl, commonly with up to 18 carbon atoms per group, or polyethylene glycol attached by ester linkages. The expression “poly (glutamic acid)” and cognate expressions herein are to be construed as covering any of the aforesaid possibilities unless the context otherwise demands.

Various substitutions of naturally occurring, unusual, or chemically modified amino acids may comprise the amino acid composition of the poly(amino acid) polymer.

Naturally occurring amino acids for use in the present invention as amino acids or substitutions of a poly(amino acid) are alanine, arginine, asparagine, aspartic acid, citrulline, cysteine, glutamine, glycine, histidine, isoleucine, leucine, lysine, methionine, ornithine, phenylalanine, proline, serine, threonine, tryptophan, tyrosine, valine, hydroxyproline, ε-carboxyglutamate, phenylglycine, or O-phosphoserine.

Non-naturally occurring amino acids for use in the present invention include, for example, β-alanine, α-amino butyric acid, γ-amino butyric acid, γ-(aminophenyl) butyric acid, α-amino isobutyric acid, citrulline, ε-amino caproic acid, 7-amino heptanoic acid, β-aspartic acid, aminobenzoic acid, aminophenyl acetic acid, aminophenyl butyric acid, γ-glutamic acid, ε-lysine, methionine sulfone, norleucine, norvaline, ornithine, d-ornithine, p-nitro-phenylalanine, hydroxy proline, 1,2,3,4,-tetrahydroisoquinoline-3-carboxylic acid, and thioproline.

Amino acid substitutions are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

Pseudo-poly (amino acids) may also be used in the present invention. Pseudo-poly(amino acids) differ from the poly(amino acids) described above in that dipeptide monomers are covalently bound through other than the normal peptide linkages. Pseudo-poly(amino acids) suitable for use in accordance with the present invention are those, for example in Kohn et al., Amer. Chem. Soc., 109:917 (1987) and Pulapura et al., J. Polymer Preprints, 31:23 (1990), each of which are incorporated herein by reference. The pseudo-poly(amino acids) can be used alone or in combination with the mixtures of classical poly(amino acids) and pseudo-poly(amino acids) in accordance with the invention.

A poly(amino acid) may, at the lower end of the amino acid substitution range, have 1, 2, 3, 4, 5, 6, 7, 8, 9, 10, 11, 12, 13, 14, 15, 16, 17, 18, 19, 20, 21, 22, 23, 24, 25 or more glutamic acid, aspartic acid, serine, tyrosine or glycine, residues, respectively, substituted by any of the naturally occurring, modified, or unusual amino acids described herein.

In other aspects of the invention, a poly(amino acid) homopolymer such as poly-glutamic acid, poly-aspartic acid, poly-serine, poly-tyrosine, poly-glycine, or a poly(amino acid) copolymer comprising a mixture of some or all of these five amino acids may, at the lower end, have about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, to about 25% of glutamic acid, aspartic acid, serine, tyrosine or glycine residues, respectively, (% by weight or by residue) and/or substituted by any of the naturally occurring, modified, or unusual amino acids described herein.

A poly(amino acid) homopolymer such as poly-glutamic acid, poly-aspartic acid, poly-serine, poly-tyrosine, or poly-glycine may, at the high end of the amino acid substitution range, have less than 25%, less than 26%, less than 27%, less than 28%, less than 29%, less than 30%, less than 31%, less than 32%, less than 33%, less than 34%, less than 35%, less than 36%, less than 37%, less than 38%, less than 39%, less than 40%, less than 41%, less than 42%, less than 43%, less than 44%, less than 45%, less than 46%, less than 47%, less than 48%, less than 49%, to less than 50% or so of the glutamic acid, aspartic acid, serine, tyrosine, or glycine residues (% by weight or by residue), respectively, substituted by any of the naturally occurring, modified, or unusual amino acids described herein. Preferably, the majority of residues comprise glutamic acid and/or aspartic acid and/or serine and/or tyrosine and/or glycine.

Polymers of the present invention have molecular weights that generally range from about 1,000 daltons to less than 10,000,000 daltons. The polymers of the present invention, in certain embodiments, have a molecular weight of about 10 daltons to about 5,000 daltons, including all integer values within this range, including, for example, 100, 200, 300, 500, 1,000, 1,500, 2,000, 2,500, 3,000, 3,500, 4,000, and 4,500 daltons, and in certain other embodiments have a molecular weight of about 600 daltons, about 32,000 daltons or about 33,000 daltons.

The poly(amino acid) polymers may be synthesized in accordance with several types of standard techniques, including chemical and recombinant processes. For example, a homopolymer of glutamic acid may be prepared in a two-step process, in which (i) glutamic acid is treated with phosgene or an equivalent reagent, e.g., diphosgene, at a temperature of from 15° C. to 70° C. to form an N-carboxyanhydride (NCA), and (ii) ring-opening polymerization of the N-carboxyanhydride is effected with a base to yield poly-(glutamic acid). Suitable bases include alkoxides, e.g., alkali metal alkoxides such as sodium methoxide, organometallic compounds and primary, secondary or tertiary amines, for example butylamine or triethylamine. See, U.S. Pat. No. 5,470,510. There are numerous other methods for chemically synthesizing poly (amino acids).

The amino acid polymers of the present invention may be produced recombinantly for example, by utilizing transformed E. coli. Limited bacterial production of poly (glutamic acid) is described, for example in EP-A-410,638. Recombinant processes conducted using bacteria will commonly yield poly (l-glutamic acid), although bacteria are known that will provide the d-form.

In some embodiments, the anti-cancer drug conjugated to the polymer is a taxane. Taxanes include paclitaxel, docetaxel and other chemicals that have the taxane skeleton. See Cortes et al., J. Clin. Oncol. 13:2643-2655 (1995). Examples of taxane compounds and methods for their preparation are set forth in U.S. Pat. No. 4,942,184.

In preferred embodiments, paclitaxel is conjugated to poly-(l-glutamic acid). The conjugated drugs of the invention are multiple taxane skeleton molecules while the unconjugated drug (e.g., traditional paclitaxel) is a single taxane skeleton molecule. To provide at least one meaningful comparison between the conjugated and unconjugated drugs, each taxane skeleton molecule in the conjugated drug is calculated as one paclitaxel (i.e., unconjugated paclitaxel) equivalent. In one embodiment, the drug conjugate, i.e., a polyglutamate paclitaxel conjugate, is a multiple taxane skeleton molecule with the chemical name 5-beta, 20-epoxy-1,2-alpha, 4,7-beta, 10-beta, 13-alpha-hexahydroxy-tax-11-en-9-one 4,10-diacetate 2-benzoate 13-ester with (2R,3S)-N-benzoyl-3-phenyl-isoserine) 2′, gamma carboxylate ester of α-poly-1-glutamic acid.

An example of a useful polyglutamate paclitaxel conjugate includes paclitaxel poliglumex (PPX; the nonproprietary name adopted by the United States Approved Name (USAN) Council (brand name Xyotax™)). PPX has an average MW in the range of about 35,000 to about 40,000 Da, but not exceeding 75,000 Da, with about 35% to about 37% weight to weight (w/w) paclitaxel loading.

In other embodiments, the anti-cancer drug or agent is camptothecin, or an analog, derivative, or prodrug thereof. Camptothecin (CPT) compounds include various 20(S)-camptothecins, analogs of 20(S)-camptothecin, and derivatives of 20(S)-camptothecin. Camptothecin, when used in the context of this invention, includes the plant alkaloid 20(S)-camptothecin, both substituted and unsubstituted camptothecins, and analogs thereof. Examples of camptothecin derivatives include, but are not limited to, 9-nitro-20(S)-camptothecin, 9-amino-20(S)-camptothecin, 9-methyl-camptothecin, 9-chlorocamptothecin, 9-flouro-camptothecin, 7-ethyl camptothecin, 10-methylcamptothecin, 10-chloro-camptothecin, 10-bromo-camptothecin, 10-fluoro-camptothecin, 9-methoxy-camptothecin, 11-fluoro-camptothecin, 7-ethyl-10-hydroxy camptothecin, 10,11-methylenedioxy camptothecin, and 10,11-ethylenedioxy camptothecin, and 7-(4-methylpiperazinomethylene)-10,11-methylenedioxy camptothecin. Prodrugs of camptothecin include, but are not limited to, esterified camptothecin derivatives as described in U.S. Pat. No. 5,731,316, such as camptothecin 20-O-propionate, camptothecin 20-O-butyrate, camptothecin 20-O-valerate, camptothecin 20-O-heptanoate, camptothecin 20-O-nonanoate, camptothecin 20-O-crotonate, camptothecin 20-O-2′,3′-epoxy-butyrate, nitrocamptothecin 20-O-acetate, nitrocamptothecin 20-O-propionate, and nitrocamptothecin 20-O-butyrate. Particular examples of 20(S)-camptothecins include 9-nitrocamptothecin, 9-aminocamptothecin, 10,11-methylendioxy-20(S)-camptothecin, topotecan, irinotecan, 7-ethyl-10-hydroxy camptothecin, or -another substituted camptothecin that is substituted at least one of the 7, 9, 10, 11, or 12 positions. These camptothecins may optionally be substituted.

Substitutions may be made to the camptothecin scaffold, while still retaining activity. In certain embodiments, the camptothecin scaffold is substituted at the 7, 9, 10, 11, and/or 12 positions. Such substitutions may serve to provide differential activities over the unsubstituted camptothecin compound. Examples of substituted camptothecins include 9-nitrocamptothecin, 9-aminocamptothecin, 10,11-methylendioxy-20(S)-camptothecin. topotecan, irinotecan, 7-ethyl-10-hydroxy camptothecin, or another substituted camptothecin that is substituted at least one of the 7, 9, 10, 11, or 12 positions.

Native, unsubstituted, camptothecin can be obtained by purification of the natural extract, or may be obtained from the Stehlin Foundation for Cancer Research (Houston, Tex.).

Substituted camptothecins can be obtained using methods known in the literature, or can be obtained from commercial suppliers. For example, 9-nitrocamptothecin may be obtained from SuperGen, Inc. (San Ramon, Calif.), and 9-aminocamptothecin may be obtained from Idec Pharmaceuticals (San Diego, Calif.). Camptothecin and various analogs may also be obtained from standard fine chemical supply houses, such as Sigma Chemicals (St. Louis, Mo.).

Aside from taxanes and camptothecins, other anti-cancer drugs that may be useful in the compositions and methods of the present invention include etopside, teniposide, fludarabine, doxorubicin, daunomycin, emodin, 5-fluorouracil, FUDR, estradiol, camptothecin, retinoic acids, verapamil, epothilones and cyclosporin. More generally, anti-cancer drugs with a free hydroxyl group that may provide a conjugation site may be used.

The invention contemplates the use of a single polymer as well as two or more different polymers. Different polymers include, for example, similar polymers of different lengths, as well as substantially different polymers. The invention includes the use of a drug conjugated to a single polymer and a drug conjugated to multiple different polymers. Similarly, the invention includes the use of two or more drugs, each conjugated to the same type of polymer, as well as mixtures of two or more drugs, each conjugated to a different polymer. In certain embodiments, two or more different drug moieties may be conjugated to a single polymer. The invention also contemplates administration of other cancer drugs (e.g., carboplatin) in addition to the drug conjugate and/or estrogen therapy.

The nature of the bond or association between the drug and the polymer is not critical. For example, a conjugated drug-polymer interaction is through a covalent bond, whereas an association may be through charge-charge interactions, Van der Waal forces, and the like. Covalent bonds may be generated synthetically or by genetic fusion to produce a recombinant polymer-drug fusion protein. Exemplary methods of conjugating a polymer to drug are described, for example, in U.S. Pat. Nos. 5,977,163, 6,262,107 and 6,441,025, and International Patent Publications WO 99/49901, WO 97/33552, WO 01/26693, and WO 01/70275.

The polymer and the drug may be conjugated or associated directly or via a secondary molecule such as a linker or a spacer (see e.g., WO 01/70275, the technology disclosed therein can be applied to any drug conjugate, particularly to polyamino acid conjugates). Preferred linkers include those that are relatively stable to hydrolysis in the circulation. Exemplary linkers include amino acids, hydroxyacids, diols, aminothiols, hydroxythiols, aminoalcohols, beta alanines, glycol and combinations thereof. In addition, the drug may require modification prior to conjugation, such as the introduction of a new functional group, the modification of a preexisting functional group or the attachment of a spacer molecule.

Chemical coupling may be achieved using commercially available homo- or hetero-bifunctional cross-linking compounds, according to methods known and available in the art, such as those described, for example, in Hermanson, Bioconjugate Techniques, Academic Press, Inc., 1995, and Wong, Chemistry of Protein Conjugation and Cross-linking, CRC Press, 1991.

Additional examples of methods for linking polymers to drugs or other molecules are described in Hoffman et al., Biol. Chem. 370:575-582 (1989); Wiesmuller et al., Vaccine 7:29-33 (1989); Wiesmuller et al., Int. J. Peptide Protein Res. 40:255-260 (1992); Defourt et al., Proc. Natl. Acad. Sci. 89:3879-3883 (1992); Tohokuni et al., J. Am. Chem. Soc. 116:395-396 (1994); Rachel, Chem. Common. 2087-2088 (1997); Kamitakahara, Angew. Chem. Int. Ed. 37:1524-1528 (1998); and Dullenkopf et al., Chem. Etc. J. 5:2432-2438 (1999).

In certain embodiments, a polymer is conjugated to a drug by chemical conjugation, as described in U.S. Pat. No. 5,977,163. In this method, polyglutamic acid conjugates are prepared as a sodium salt, dialyzed to remove low molecular weight contaminants and excess salts, and then lyophilized. Other chemical conjugation methods are described in WO 01/26693 A2. According to this method, a polyglutamic acid polymer is covalently bonded to a drug by a direct linkage between a carboxylic acid residue of the polyglutamic acid and a functional group of the drug, or by an indirect linkage via one or more bifunctional groups. Other methods are disclosed in March, Advanced Organic Chemistry, Wiley Interscience, 4th ed., 1992.

In one embodiment, a polyglutamate carrier is coupled to a drug according to a method as follows:
(a) providing a protonated form of a polyglutamic acid polymer and a drug for conjugation thereto;
(b) covalently linking said drug to said polyglutamic acid polymer in an inert organic solvent to form a polyglutamic acid-drug conjugate;
(c) precipitating said polyglutamic acid-drug conjugate from solution by addition of an excess volume of aqueous salt solution; and
(d) collecting said conjugate as a protonated solid.

The protonated form of the polyglutamic acid polymer in step (a) is obtained by acidifying a solution containing the salt of the polyglutamic acid to be used as a starting material, and converting the salt to its acid form. After separating the solid by centrifugation, the solid is washed with water, the polyglutamic acid is then dried, preferably by lyophilization and preferably to a constant weight comprising between about 2% to about 21% water, between about 7% to about 21% water, or between 7% and 21% water, prior to conjugation to a desired drug (b).

Conjugates may be produced in whole, or in part, using recombinant DNA technology. For example, a polymer or drug, or both, may be produced by recombinant means and thereafter associated or conjugated. Alternatively, a single polypeptide, for example, comprising both the polymer and the drug may be produced as a fusion protein. Methods of constructing recombinant expression vectors are known in the art, as are methods of expressing recombinant polypeptides in a variety of organisms, such as bacteria and yeast.

The amount of drug conjugated to the polymer is variable. At the lower end, the drug polymer conjugate may comprise from about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21% about 22%, about 23%, about 24%, to about 25% (w/w) of drug relative to the mass of the conjugate. At the high end, the drug-polymer conjugate may comprise from about 26%, about 27%, about 28%, about 29%, about 30%, about 31% about 32%, about 33%, about 34%, about 35%, about 36%, about 37%, about 38%, about 39%, about 40%, to about 50% or more (w/w) of drug relative to the mass of the conjugate.

Similarly, the number of molecules of drug conjugated per molecule of polymer can vary. At the lower end, the drug-polymer conjugate may comprise from about 1, about 2, about 3, about 4, about 5, about 6, about 7, about 8, about 9, about 10, about 11, about 12, about 13, about 14, about 15, about 16, about 17, about 18, about 19, to about 20 or more molecules of the drug per molecule of polymer. At the higher end, the drug-polymer conjugate may comprise from about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60 about 61, about 62, about 63, about 64, about 65, about 66, about 67, about 68, about 69, about 70, about 71, about 72, about 73, about 74, to about 75 or more molecules or more of drug per molecule of polymer.

A polymer may be associated with one or more discrete or overlapping sites on a drug molecule. Conversely, in certain embodiments, a drug molecule may be associated with one or more discrete sites on a polymer. Accordingly, in certain embodiments, compositions of the invention include polymers associated with drugs through different sites on the drug, as well as drugs associated with a polymer through different sites on the polymer. Different linkers may be used to direct association through different sites, or a single linker may be used, depending on the particular functional groups present at each site. In certain embodiments, the invention includes a composition comprising a mixture of one or more polymers associated with one or more drugs through one or more different or overlapping sites on each drug or polymer.

Drug conjugates of the present invention may be delivered or administered by any therapeutically suitable means. For example, administration may be parenteral or oral. In preferred embodiments administration is intravenous (i.v.).

Compositions include the conjugate and a pharmaceutically acceptable carrier. As used herein, pharmaceutically acceptable carriers include solvents (e.g., buffered aqueous medium), dispersion media, coatings, antibacterial and antifungal agents, isotonicity agents (e.g., glucose) and the like. Pharmaceutical compositions containing the conjugates suitable for intravenous use include sterile aqueous solutions or dispersions, as well as sterile powders that can then be reconstituted in an aqueous solution. The carrier may be a solvent or dispersion medium containing, for example, water, ethanol, a polyol, suitable mixtures thereof, and vegetable oils. Suitable injectable solutions may be prepared by incorporating an appropriate amount of the conjugate into an appropriate solvent, optionally with various other ingredients as listed above. Solutions may be sterilized by filtration. Dispersions may be prepared by incorporating the conjugate into a sterile vehicle containing the dispersion medium and any other ingredients from those listed above. Sterile powders may be prepared by vacuum drying and freeze-drying techniques, yielding a powder of the conjugate and any additional desired ingredients from a previously sterile-filtered solution thereof.

Pharmaceutical compositions may be delivered or administered in an appropriate dosage and for a suitable duration and frequency. Dose and duration of administration are typically determined by such factors as the condition of the patient, the type and severity of the patient's disease, the particular form of the active ingredient and the method of administration. The appropriate dosage and treatment regimens are designed to achieve a therapeutic benefit (e.g., an improved clinical outcome, such as more frequent complete or partial remissions, or longer disease-free and/or OS). Examples of different ranges of dosage and administration schedules are provided in U.S. Pat. No. 5,670,537 (disclosing dosages and administration schedules for taxol).

In some embodiments of the invention, patients in need of such treatment receive a drug conjugate such as PPX, on a bi-weekly or other clinically useful schedule, at dose levels typically used for the particular conjugate involved. To provide a meaningful comparison between multiple taxane skeleton molecule therapies and other single taxane skeleton molecule therapies, the dosing of taxane conjugates can be expressed as “paclitaxel equivalents”. For example, for dosing purposes, each taxane skeleton in a taxane conjugate molecule is calculated as one paclitaxel equivalent. The dose level generally ranges from about 175 to about 250 milligrams/meter squared (mg/m²) paclitaxel equivalents. In preferred embodiments, patients are treated with PPX in an amount of about 200 to about 250 mg/m² paclitaxel equivalents every 21 days or about once every 3 weeks. In the most preferred embodiment patients are treated with PPX in an amount of about 175 mg/m² paclitaxel equivalent every 21 days or about once every 3 weeks. In other embodiments, patients are treated with a taxane conjugate, such as PPX, in an amount in excess of about 250 mg/m² paclitaxel equivalents every 21 days or even in excess of about 275 mg/m² paclitaxel equivalents every 21 days.

Intravenous infusion may be for any appropriate time period, which is readily determinable by one of ordinary skill in the art. For example, infusions may last for about one to about 24 hours, although shorter or longer infusion times also fall within the scope of the invention.

Some embodiments of the invention contemplate the use of estrogen therapy in combination with the drug conjugate. Estrogen therapy, as used herein, refers to administration of an estrogenic substance that will increase levels of estrogen in the patient or results in a higher accumulation of the anti-cancer drug in the cancerous tissues, e.g., lung, compared to non-cancerous tissue (which in the case of lung cancer, includes bone and liver). Thus, as used herein, “estrogen” includes, but is not limited to, any of the naturally occurring mammalian estrogens and congeners thereof, e.g., estradiol and its organic esters (e.g., estradiol benzoate, estradiol cypionate and estradiol valerate), estrone, diethylstilbestrol, piperazine estrone sulfate, ethinyl estradiol, mestranol, polyestradiol phosphate, estriol, estriol hemisuccinate, quinestrol, estropipate, pinestrol, estrone potassium sulfate, and tibolone, estrogen metabolites, e.g., 17-β-estradiol, estrogen analogs, natural compounds with estrogenic activity, e.g., phytoestrogens, such as genestein or isoflavone, estrogen agonists, e.g., selective estrogen receptor modulators with some agonistic activity (e.g., tamoxifen), and other substances capable of interaction with cell surface estrogen receptors. These estrogens and estrogenic substances may be natural or synthetic.

Estrogen or estrogenic substances can be formulated by any means compatible with mammalian physiology and the selected route of delivery. These methods are well known in the art. See U.S. Pharmacopeia National Formulary, United States Pharmacopeal Convention, Inc., pp. 530-541 (1990).

Estrogen therapy can be administered by any suitable route such as locally, orally, systemically, intravenously, intramuscularly, mucosally, or transdermally (e.g., a patch). The estrogen therapy may be administered on a regular (e.g., a daily/weekly) basis, and may be intermittent or substantially continuous with respect to the drug-conjugate therapy. Typical dose ranges depend on the compound and the characteristics of the patient. The daily dose of the estrogen is typically based on the amount of a given preparation necessary to maintain serum or plasma estrogen levels equal to or greater than 30 pg/ml when estrogen or its metabolites are used.

Treatment regimens include, but are not limited to, hormone replacement therapy, such as estrogen/progesterone therapy at 0.3 mg/1.5 mg, 0.45 mg/1.5 mg, 0.625 mg/2.5 mg, or 0.625 mg/5.0 mg daily. In another embodiment, therapy may comprise estrogen therapy at 0.15 mg, 0.3 mg, 0.625 mg or 1.25 mg once or twice daily. In a preferred embodiment, therapy comprises 10 mg estrogen three times daily. An example of estrogen is Premarin® (Wyeth Pharmaceuticals, Inc., PA). In some embodiments, the estrogen/progesterone combination is accomplished by administration of Prempro® (Wyeth Pharmaceuticals, Inc., PA). Alternate therapies include tamoxifen administered at 20 to 40 mg per day in 10 mg doses multiple times per day or 20 mg daily in a single dose.

The methods of the invention do not require that each component of the combination therapy is delivered by the same route or even at the same time. In some embodiments of the invention, however, the drug conjugate and estrogen therapy are given at the same time by the same route of administration. In a more preferred embodiment, the estrogen therapy is administered transdermally (e.g., by a patch) or orally (e.g., daily tablet, capsule or pill) so as to be substantially continuous over the duration of the drug conjugate treatment course.

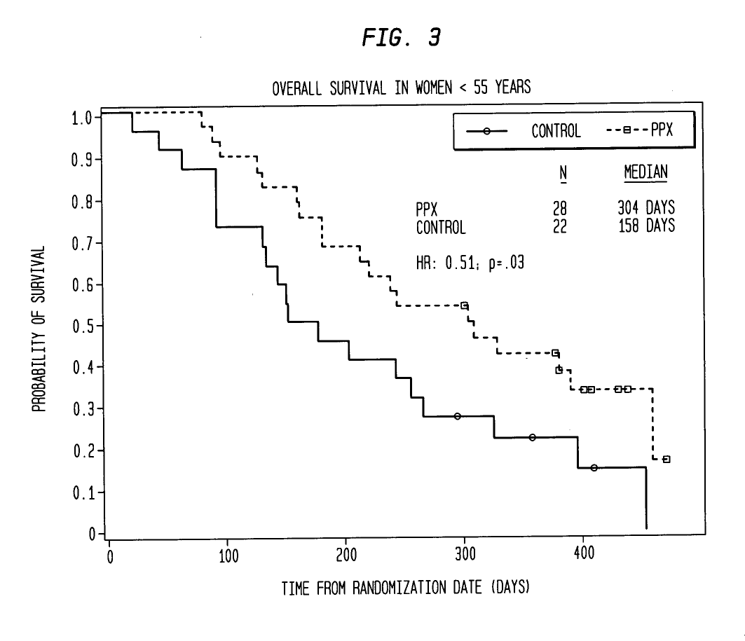

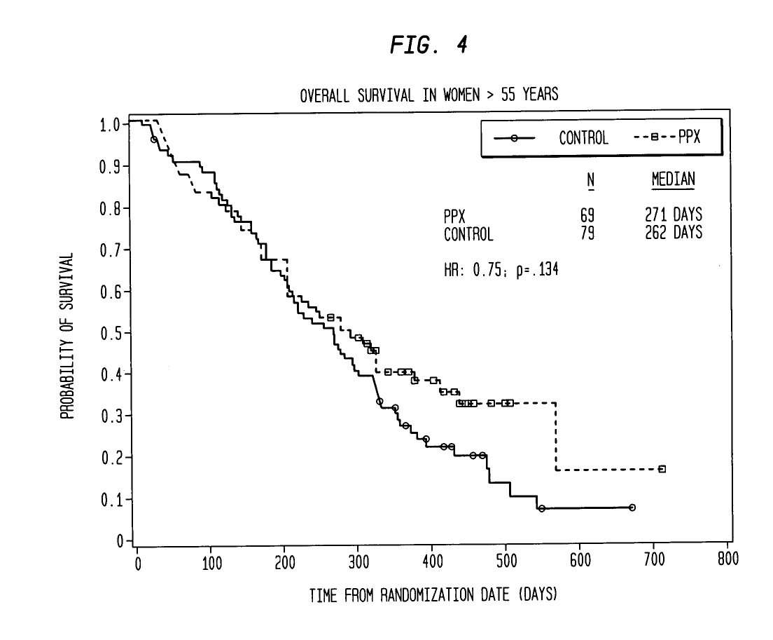

The present invention is also directed to a method of selecting or choosing a cancer treatment based on blood serum or plasma estrogen levels. Patients with premenopausal estrogen levels are more likely to respond favorably to treatment with a conjugated form of the anti-cancer drug, e.g., PPX, while those patients with low (e.g., postmenopausal) estrogen levels, can be treated with PPX or another cancer therapy such as unconjugated paclitaxel (see examples 1 and 2, especially FIGS. 3 and 4).

The method for selecting or choosing a cancer treatment entails measuring the estrogen level in the serum or plasma of a blood sample obtained from a cancer patient, and correlating the estrogen level with a treatment option. Premenopausal estrogen levels are correlated with a cancer treatment comprising the administration of PPX. Postmenopausal estrogen levels are correlated with a cancer treatment that can include either PPX or paclitaxel. The method is particularly useful in connection with estrogen receptor bearing cancers, which, as previously described, includes any cancer characterized by the presence of cancer cells bearing estrogen receptors, or a cancer that originates in an organ or tissue containing normal (non-diseased) cells that bear estrogen receptors, wherein the estrogen receptor may be transiently expressed and/or the estrogen receptor bearing cells represent a fraction of the total population of cells.

In order to fully illustrate the present invention and advantages thereof, the following specific examples are given, it being understood that the same is intended only as illustrative and in no way limitative.

### EXAMPLE 1

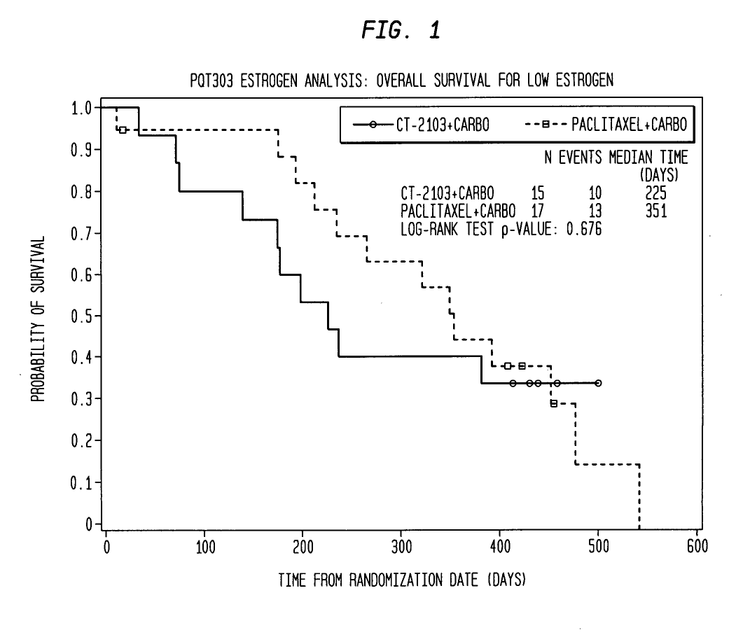

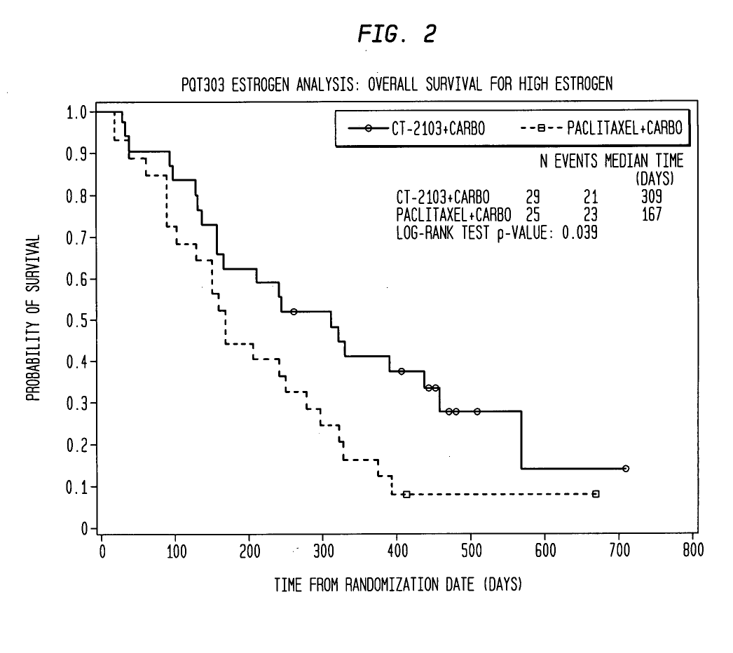

The experiments and results presented in example 1 describe the correlation between estrogen levels and treatment with CT-2103 versus paclitaxel, as measured by overall survival (OS). The data depicted in FIGS. 1 and 2 were derived from a multinational, phase III, open-label study. Initially, 400 patients with NSCLC were randomized equally to 1 of 2 treatment arms: 199 patients in Arm 1 (CT-2103, i.e., PPX) at a dose of 210 mg/m² in combination with carboplatin (AUC 6); 201 patients in Arm 2 (unconjugated Paclitaxel) at a dose of 225 mg/m² in combination with carboplatin (AUC 6). Randomization was stratified to minimize potential imbalances between the 2 treatment arms. After they were selected for the study, patients were stratified by disease stage (IV vs other), gender, geographic location (US vs. Western Europe and Canada vs. rest of the world), and history of brain metastases (yes vs. no).

From the initial sample set, a subset consisting of either high or low estrogen women was analyzed. From Arm 1, 29 high estrogen and 15 low estrogen women were studied and from Arm 2, 25 high estrogen and 17 low estrogen women were studied. Estrogen levels in women were determined by a competitive immunoassay (e.g., DPC IMMULITE 2000 Analyzer, NY; DPC IMMULITE Analyzer, Ireland). Comparisons were made between Arms 1 and 2 for the low estrogen group (FIG. 1) and between Arms 1 and 2 for the high estrogen group (FIG. 2).

Inclusion criteria consisted of:
1. a histologically or cytologically confirmed diagnosis of NSCLC;
2. meeting one of the following criteria for disease status
   a. locally advanced or recurrent disease previously treated with radiation and/or surgery,
   b. stage IIIB patients who were not candidates for combined modality therapy, or
   c. patients with stage IV disease;
3. an ECOG performance score of 2; and
4. adequate bone marrow, renal, and hepatic function. In addition, patients with known brain metastases must have received standard antitumor treatment (no systemic chemotherapy combined with radiation); patients must have adequately recovered from therapy and displayed stable neurologic function for 2 weeks before randomization.

Patients received CT-2103/carboplatin or paclitaxel/carboplatin, by intravenous infusion, on day 1 of each 21-day cycle for a total of 6 cycles. Toxicities were evaluated at each patient visit. Hematology, clinical chemistry, and coagulation parameters were evaluated before and during the study period. Tumor burden was evaluated according to Response Criteria in Solid Tumors (RECIST); disease-related symptoms were measured by the functional assessment cancer therapy-lung cancer score (FACT-LCS), and computerized tomography (CT) scans were evaluated at regular intervals. Patients were withdrawn for disease progression, intolerable toxicity, patient withdrawal of consent, or investigator decision to withdraw the patient. An end-of-treatment assessment was performed 22-26 days after completion of the final study treatment; patients were also contacted at 8-week intervals following the end-of-treatment visit to obtain additional survival and disease status information. The efficacy and safety outcomes of this study were monitored by an independent Data Monitoring Committee (DMC).

Efficacy of the treatments was determined by evaluation of tumor burden according to RECIST. In addition, CT scans were performed at baseline and in the 3^{rd} week of every even-numbered cycle. Disease-related symptoms were measured by the functional assessment cancer therapy-lung cancer score (FACT-LCS) within 3 days before each study treatment.

A log rank statistical analysis was undertaken to compare Arm 1 and Arm 2 in the high estrogen women and Arm 1 and Arm 2 in the low estrogen women.

Results from the above study indicated no statistically significant difference in the OS of postmenopausal (i.e., low estrogen) women diagnosed with NSCLC and treated with either CT-2103+carboplatin or paclitaxel+carboplatin (FIG. 1). In stark contrast, there was a statistically significant increase in survival when CT-2103+carboplatin was given to post-pubescent, premenopausal women with NSCLC (i.e. high estrogen) versus. paclitaxel+carboplatin (FIG. 2). The statistically significant difference for the results of the study depicted in FIG. 2 was even more surprising given the low sample size.

These results suggest that women diagnosed with NSCLC, and possessing premenopausal estrogen levels, would benefit from a cancer treatment that included CT-2103 (i.e., PPX), while women with postmenopausal estrogen levels can be treated with either CT-2103 or paclitaxel.

### EXAMPLE 2

The experiments and results presented in example 2 describe the correlation between estrogen levels, gender (i.e., sex), and treatment with PPX versus paclitaxel, as measured by overall survival (OS). Two phase III studies, enrolling a total of 781 patients with NSCLC and a poor performance status (PS2), were conducted. In one study (STELLAR 3), patients were divided into two treatment groups, the experimental group received PPX (210 mg/m² plus carboplatin AUC 6, every 3 weeks) and the control group received paclitaxel (225 mg/m² plus carboplatin AUC 6, every 3 weeks). In the other study (STELLAR 4), the experimental group received PPX (175 mg/m², every 3 weeks) and the control group received gemcitabine (1000 mg/m² on days 1, 8, and 15, every 4 weeks) or vinorelbine (30 mg/m² on days 1, 8, and 15, every 3 weeks). In order to study the effect of sex and estrogen on survival among the various treatment groups, the results from both phase III clinical trials were combined and a composite analysis performed.

The results of the effect of sex on survival outcome are presented in Table 1 below. These results indicated that a statistically greater number of females treated with PPX reached the one-year survival point than females in the control group. On the other hand, there was no such difference in one-year survival for males in the PPX group vs. males in the control group.

TABLE 1

| | |
|---|---|
| FEMALE | MALE |

| | PPX | CONTROL | PPX | CONTROL |
|---|---|---|---|---|
| ^{a}N | 97 | 101 | 283 | 290 |
| OS | 285 | 233 | 220 | 207 |
| (DAYS) | | | | |
| 1-YR | 40% | 25% | 26% | 30% |

| | | |
|---|---|---|
| HR | 0.70 | 1.05 |
| p-VALUE | .03 | .585 |

^{a}N: the number of patients in a given group

To evaluate the effect of estrogen on survival in women treated with PPX, the data were analyzed retrospectively by menopausal status. Functional menopausal status was assigned based on age and pretreatment estradiol levels, when available. The study was a composite of results from both clinical studies. In women under 55 years of age, OS was prolonged for patients receiving PPX compared to those in the control arm (FIG. 3). In women over 55 years of age, OS was similar between treatment groups (FIG. 4).

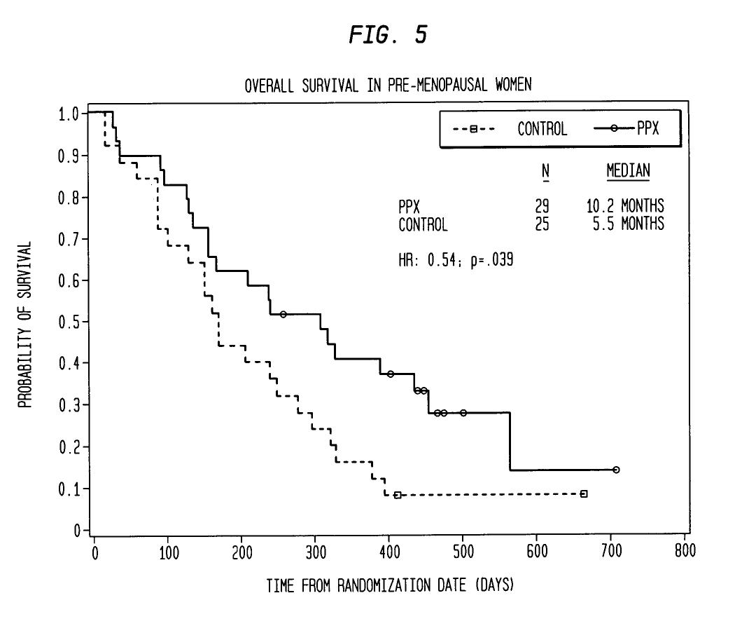

In STELLAR 3, estrogen levels were available for some women. Women with premenopausal estrogen levels had an improved outcome when treated with PPX compared to control (FIG. 5). By contrast, the treatment group had no apparent effect on survival in patients with postmenopausal estrogen levels (data not shown).

### EXAMPLE 3

The experiments and results presented in example 3 describe the mechanism by which PPX is brought into cells. Cellular uptake of PPX was studied, in vitro, in two cell lines obtained from the American Type Culture Collection (ATCC), NCI-H460 human lung carcinoma (H460; ATCC HTB-177) and RAW 264.7 mouse monocyte/macrophage (RAW; ATCC TIB-71), which were cultured and treated independently, with PPX.

In one set of experiments, PPX uptake was measured utilizing radiolabled PPX, which was made as follows: Radiolabeled PPX was made by conjugating a standard preparation of poly-L glutamic acid with paclitaxel that was uniformly labeled with ¹⁴C in the 2-benzoyl ring (¹⁴C-PPX; Sigma-Aldrich, 1.762 microcuries (μCi)/mg specific activity). Cells were treated with 0.01-10 μM ¹⁴C-PPX for 2 minutes or 3-4 hours. Radioactivity was then quantitated in the media by scintillation counting. Harvested cells were extracted in ethyl acetate and the upper organic phase (containing unconjugated paclitaxel and low molecular weight metabolites [i.e., mono & diglutamyl paclitaxel]) and the lower aqueous phase (containing glutamyl-paclitaxel metabolites and intact PPX) was counted.

To study the distribution of PPX, RAW cells were treated with 1 μM or 10 μM of radiolabeled PPX (¹⁴C-PPX). At 2 minutes, and 3 or 4 hours, samples were taken. Radioactivity was quantitated as previously described. The results, from 3 samples, were expressed as disintegrations per minute (DPM)±the standard deviation.

The results presented in Table 2 (below) indicate a statistically significant increase (p<0.001) of intact PPX in cells treated for 3 hours with 10 μM of ¹⁴C-PPX.

TABLE 2

| | [¹⁴C-PPX] | | | |
|---|---|---|---|---|
| | 1 μM | | 10 μM | |
| | Incubation Time | | | |
| | 2 minutes | 4 hours | 2 minutes | 3 hours |
| Samples | DPMs +/− Standard Deviation, N = 3 (% control) | | | |
| Starting media | 252989 ± 4366 | 252989 ± 4366 | 2490999 ± 16716 | 2490999 ± 16716 |
| Aqueous cell | 196 ± 11 | 1980 ± 197 | 454 ± 66 | 2554 ± 90 |
| layer extract | (0.077%) | (0.783%) | (0.018%) | (0.103) |
| Organic cell | 323 ± 360 | 693 ± 410 | 207 ± 29 | 275 ± 50 |
| layer extract | (0.128%) | (0.273) | (0.008%) | (0.011%) |

In another experiment, two cohorts, of either RAW or H460 cells, were treated with ¹⁴C-PPX for 2 minutes, or 3 hours. One of the cohorts was additionally treated with an inhibitor of energy dependent endocytosis (20 μM of cytochalasin D {Cyto D, EMD Biosciences} or 1 μM Phenyl Arsine Oxide {POA, Sigma}). Radioactivity was quantitated as previously described. DPMs were calculated by subtracting the DPMs in the 2 minute incubation from the DPMs in the 3 hour incubation in the aqueous phase of the ethyl acetate extracted cell layer compartment.

The results, presented in Table 3 (below), indicate that pre-treatment of cells with an inhibitor of endocytosis results in a statistically significant decrease in ¹⁴C-PPX as measured in the aqueous phase (intact PPX).

TABLE 3

| | [¹⁴C-PPX] | | | |
|---|---|---|---|---|
| | 1 μM | | 10 μM | |
| | +/− Inhibitor | | | |
| | − | + | − | + |
| Inhibitor/Cell Type | DPMs +/− Standard Deviation, N = 3 | | | |
| Cyto D/RAW cells | 1784 ± 197 | 808 ± 106 | 2100 ± 112 | 1318 ± 57 |
| PAO/H460 cells | 73 ± 15 | 54 ± 6 | 217 ± 6 | 128 ± 11 |

DPMs were calculated by subtracting the DPMs in the 2 min incubation from the DPMs in the 3 hour incubation in the aqueous phase of the ethyl acetate extracted cell layer compartment.

The results from the ¹⁴C-PPX uptake studies indicated that intact PPX was taken up into the cellular compartment of RAW cultured macrophages and H460 tumor cells by energy-dependent endocytosis, in a dose- and time-dependent manner.

In another set of experiments, PPX uptake was studied by indirect immunfluorescence. An anti-PPX monoclonal antibody (MAb) was prepared as follows: The Plasmodium falciparum (P. falciparum) peptide (GGG GGA GLL GNV STV LLG GV; Genemed Synthesis, Inc.) was conjugated to PPX (CTI; lot# 1116-91) via a diisopropyl carbodiimide reaction in order to render the PPX molecule immunogenic. The P. falciparum peptide-PPX antigen was used to produce a MAb via the proprietary Rapid Prime® immunization procedure (ImmunoPrecise Antibodies Ltd.). Positive hybridoma clones were selected using a solid phase ELISA with immobilized P. falciparum peptide-PPX antigen. One hybridoma clone, CT-2D5, was expanded via intraperitoneal (i.p.) injection in BALB/c mice. CT-2D5 was purified from ascites fluid by Protein G chromatography. Binding affinity was assessed by solid phase ELISA methodology. MAb isotypes were determined to be IgG-2b with an Instant ChekTM-ISOTYPE kit (EY Laboratories Ltd.). The binding epitope of CT-2D5 was mapped using a competitive ELISA methodology.

After confluent cell layers were incubated with 10 nM to 10 μM of PPX for 4 hours, the cells were then fixed and processed for immunofluorescence by the following method: Fixed cells were stained with CT-2D5, the 10° antibody (Ab), and a 1:5000 dilution of AlexaFluor488 labeled goat anti-mouse F(ab′)₂ 2° Ab (Molecular Probes). Images were acquired with a Nikon Diaphot fluorescent microscope and CoolSnap HQ camera. Metamorph image processing software (Metamorph v.6.2 r6) was utilized to process and store the images.

RAW cell layers were processed as described and then co-stained with an anti-early endosomal antigen-1 Ab (EEA-1, Santa Cruz Biotechnology). H460 cell layers were processed as described. An additional cohort of H460 cell layers were co-stained with Hoechst nuclear dye and anti-α-tubulin Ab.

The results from the indirect immunofluorescent studies indicated that PPX immunostaining co-localized with an endosomal marker (data not shown). Endosomes fused with lysosomes, exposing PPX to lysosomal enzymes that metabolize the drug, principally cathepsin B.

### EXAMPLE 4

The experiments and results presented in example 4 describe the in vivo trafficking of PPX. Thus, the distribution of PPX was studied in tumor bearing, PPX treated female nude mice. Female nude mice were subcutaneously (s.c.) implanted with H460 human tumor fragments in the left flank. When the tumor mass reached a size of 100-150 mm3, mice were treated with a single i.v. dose of PPX (90 mg/kg as PTX equivalent). Mice were euthanized 24 hours post-treatment. Lung, liver, spleen and tumor samples were collected, fixed in 10% buffered formalin and paraffin embedded. Tissue sections (4 micrometers (μm)) were stained with several antibodies: CT-2D5 (previously described in example 3), CD45 rat anti-mouse (BD Pharmingen, Cat No: 55039, Clone:30-F11), F4/80 rat anti-mouse (Novus Biologicals, Cat NB 600-404 Clone CI:A3-), MHCII (HLA) mouse anti-human monoclonal Ab HLA-DR α-Chain (Dako Cytomation, Clone TAL.1B5, REF: M 0746, LOT: 00006515), Lysozyme polyclonal rabbit anti-human (Dako Cytomation, EC 3.2.1.17, Code A 0099), and myeloperoxidase (MPO) polyclonal rabbit anti-human (Dako Cytomation, Code NP082).

CT-2D5 staining was performed using Dako-ARK mouse-on-mouse kit. CD45 and F4/80 staining were performed using Vectestain ABC-Alkaline-Phosphatase kit. MHCII, Lysozyme and MPO staining was performed using Vectestain ABC-Peroxidase kit. In order to detect nonspecific staining, negative control sections were prepared as follows. During immunostaining with CT-2D5, CD45, F4/80 and MHCII negative control sections were incubated with normal blocking serum as the primary Ab. During immunostaining with Lysozyme and MPO, negative control sections were incubated with an irrelevant rabbit polyclonal primary Ab.

Histological examinations were performed using a light microscope with an attached digital camera. Microscopic examinations were performed blindly. Intensity of positive immuno reaction was scored following an arbitrary scale (from minimal to marked). Semi-quantitative evaluation of IHC positivity rate was performed by counting positive cells at high magnification (400×) in 10 fields randomly chosen. Representative fields at 100× were captured to illustrate the positivity distribution in the tissue.

Strong intra-cytoplasmic staining was found in organs of the reticuloendothelial system 24 hours after treatment with PPX; in liver, approximately 20 cells per field (cpf) with morphology compatible with Kupffer cells were strongly positive; hepatocytes appeared negative; in spleen, cells with macrophage morphology were positive (≈10-15 cpf) whereas lymphoid follicles were negative; in lung, positive cells (<1 cpf) were morphologically compatible with type II pneumocytes.

In tumor tissue, a strong intracytoplasmic staining was found in tumor capsule (≈2 cpf); morphology of positive cells was consistent with that of infiltrating macrophages. The rim between viable cells and necrosis was characterized by moderate to strong granular, extracellular, and occasionally intracellular (intracytoplasmic and intranuclear) staining. The tissue architecture in this area was severely compromised by necrotic processes, thus, a reliable identification by morphological analysis of the CT-2D5 positive cells was not possible.

In the spleen almost every cell in red pulp showed strong intracytoplasmic positivity for CD45 and slight to moderate positivity was found in white pulp too; moderate intracytoplasmic staining for F4/80 was seen in the red pulp in a lower number of cells (≈10-15 cpf). In the liver, slight to moderate intracytoplasmic staining was seen for both CD45 and F4/80 (≈20 cpf). In the lung, moderate to strong intracytoplasmic staining was seen for CD45 (≈2-5 cpf); slight intracytoplasmic staining for F4/80 was seen in few cells (<1 cpf). The morphology and distributive pattern of F4/80 positive cells in liver, spleen and lung was consistent with that CT-2D5 positive cells in the same tissues.

In the tumor, strong intracytoplasmic staining was seen for both CD45 and F4/80 (≈6 and 4 cpf, respectively) in tumor capsule. Rare CD45 and F4/80 positive cells were localized within the viable tumor tissue. The rim between viable tumor cells and necrosis was characterized by moderate to strong extracellular granular staining, and occasionally intracellular, for CD45. Morphology of these cells was consistent with that of karyolitic-karyorrhectic granulocytes. Necrotic area was characterized by marked positivity for CD45. Peri-necrotic and necrotic areas were negative for F4/80.

Specific immunoreactivity (≈10 cpf) for MHCII was localized in the external capsule of the tumor. Rare (≦1 cpf) and scattered positive cells were found in the viable portions of the tumor. Morphological analysis of immunoreactive cells indicates that were of histiocytic origin. No positive staining was found in the necrotic area.

Positive immunoreactivity for Lysozyme was localized in the external capsule of the tumor (≈10 cpf). Intense positive staining (fine granular debris) was found in the necrotic areas of the tumor.

Numerous MPO immunoreactive cells morphologically consistent with karyolitic-karyorrhectic granulocytes were seen in the necrotic areas of the tumor.

The results of this study confirm that 24 hours after injection, uptake and accumulation of PPX can be detected in different organs and in the tumor tissue.

Morphological evaluation, supported by the colocalization of CD45 (common leucocyte antigen) and F4/80 (specific murine macrophage antigen)-signal, demonstrated that CT-2D5 positive cells were all of myelo-histiocytic origin in liver, spleen, lung, and tumor capsule. These confirm that PPX was largely taken up by tissue-associated macrophages in the reticuloendothelial system without significant distribution to hepatocytes, alveolar lining cells in the lung, or the white pulp of the spleen.

Evaluation of CT-2D5 positivity in the perinecrotic and necrotic area was complicated by the limited number of viable cells. Nonetheless, CD45, MPO and lysozyme positivity and morphologic examination suggest that PPX accumulation in these areas of tumors is associated with polymorphonuclear (PMN) leucocytes infiltration and intracellular PPX is due to their phagocytic activity in that area.

The lack of accumulation of macrophages in the central portion of this tumor model prevents assessment of the potential interaction of PPX with Tumor Associate Macrophages (TAMs) both as a potentially important source of intratumoral PPX metabolism and potential effects of high and prolonged concentrations of intracellular free paclitaxel PPX on TAM function.

Accumulation of PPX detected in the perinecrotic area of H460 tumor xenograft tumor tissue is compatible with enhanced distribution of PPX through the enhanced permeability and retention (EPR) effect of tumor vasculature.

The presence of high levels of PPX in the tumor capsule of H460 xenografts is consistent with prior biodistribution studies and confirms the preferential tumor accumulation of PPX.

Intracellular localization of PPX is observed primarily in phagocytotic cells, i.e., tissue-associated macrophages (liver, spleen) or tumor-infiltrating macrophages.

In tumor tissue, intra- and extra-cellular PPX co-localizes with a marker for lysosomal activity.

Together, these observations are consistent with a model in which PPX preferentially accumulates in tumor tissue followed by the release of paclitaxel through the proteolytic activity of lysosomal enzymes.

### EXAMPLE 5

A study was conducted to examine the effect of estrogen on cathepsin B and PPX activity in human tumor xenografts. To study this effect, HT-29 human colorectal cancer (CRC) and H460 human non-small lung cancer (NSLCL) (both from the ATCC) cells were transplanted s.c. into female CD nu/nu mice. Placebo or 0.17, 0.36, 0.72 mg/60 days of 17β Estradiol (E2) controlled release pellets (IRA) were implanted s.c. on day 0 and after 5 days human tumor fragments were transplanted. On day 5, 21, 28 and 35 after pellet implant, blood and tumors were collected (5 mice/time point).

The effects of estrogen treatment on PPX and paclitaxel antitumor activity on the HT-29 human CRC xenograft tumor model was analyzed by the following method: when tumors were 120-150 mg (i.e., advanced tumors), PPX (i.v./qd1 90 mg/kilogram (kg) PTX equivalents) and PTX (i.v./qd1 90 mg/kg, Sigma Aldrich), were administered to tumor and placebo (P) or E2 pellet bearing mice. One day (24 hours) and 7 days (168 hours) after drug treatment, tumors were collected and were fast frozen in liquid nitrogen immediately after dissection.

Antitumor activity parameters were determined as follows:
1. Tumor weight inhibition % (TWI %) equals 100 minus (TW of treated divided by TW of control) times 100, which was evaluated at day 60 from pellet implant.
2. Tumor growth delay (TGD): TGD equals TG Treated minus TG Control, where TG is the mean time, in days, for the tumor to reach a weight of 1 gram (g).
3. Log cell kill (LCK) was calculated by the formula: LCK equals ((TGD 1 g) divided by 3.32) times DT, where DT is the time (days) necessary for the tumor to double in volume, in control and experimental groups.

Statistical analyses were performed using one-way ANOVA Bonferroni post test.

Estrogen plasma levels were determined by a RadioImmunoAssay KIT (DiaSorin).

Analysis of unconjugated and total paclitaxel in tumor tissues was determined by the following method: after appropriate homogenization of the tissues, the unconjugated paclitaxel concentrations were determined through liquid/liquid extraction with methyl-terbutyl ether (MTBE). The total paclitaxel was measured after methanolysis reaction of PPX followed by MTBE extraction of the released paclitaxel. The paclitaxel content in the processed samples was assayed by LC/MS/MS under optimized conditions in selected reaction monitoring.

ERα, ERβ, E-Cadherin and Id-2 gene expression was determined by RT-PCR, relating their RNA expression to that of GAPDH gene. Total RNA extracted from tumors was reversely transcribed by Thermoscript RT-PCR System (INVITROGEN). The cDNA generated was used as template for PCR with primers specific for ERα and β, E-Cadherin and Id-2.

Western blot analysis was performed as follows: proteins were extracted by homogenization in RIPA buffer and their content was determined by Bio-Rad Protein Assay. Protein aliquots were separated by size on 10% SDS-PAGE and transferred to nitrocellulose membrane. Immunodetection was performed using: anti-ERa Polyclonal Ab (HC-20, Santa Cruz Biotechnologies), anti-ERα Polyclonal Ab (06-629, Upstate Biotechnologies), anti-α-Actin (Sigma), and anti-α-Tubulin (Santa CruzBiotechnologies).

Cathepsin B activity assay was performed as follows: proteins were extracted by homogenization in cathepsin B buffer in ratio of 1 ml of buffer per 100 mg of tissue. The test utilized the ability of cathepsin B to digest the synthetic substrate Z-Arg-Arg-AMC. Released AMC was determined fluorometrically at excitation wavelength 390 nm and emission wave-length 460 nm. The activity of cathepsin B was quantified versus an AMC standard curve.

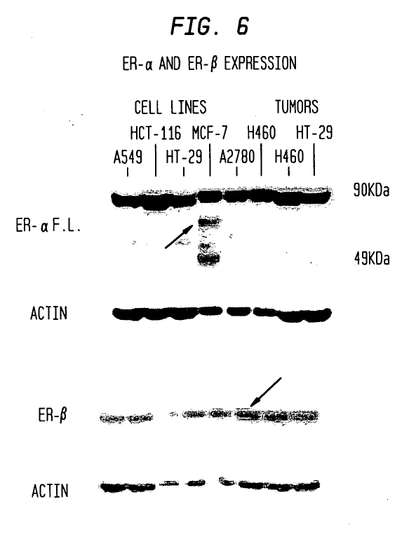

The ERα and ERβ expression in human tumor cell lines and the HT-29 and H460 human xenograft tumor model was demonstrated via western blot analysis (FIG. 6).

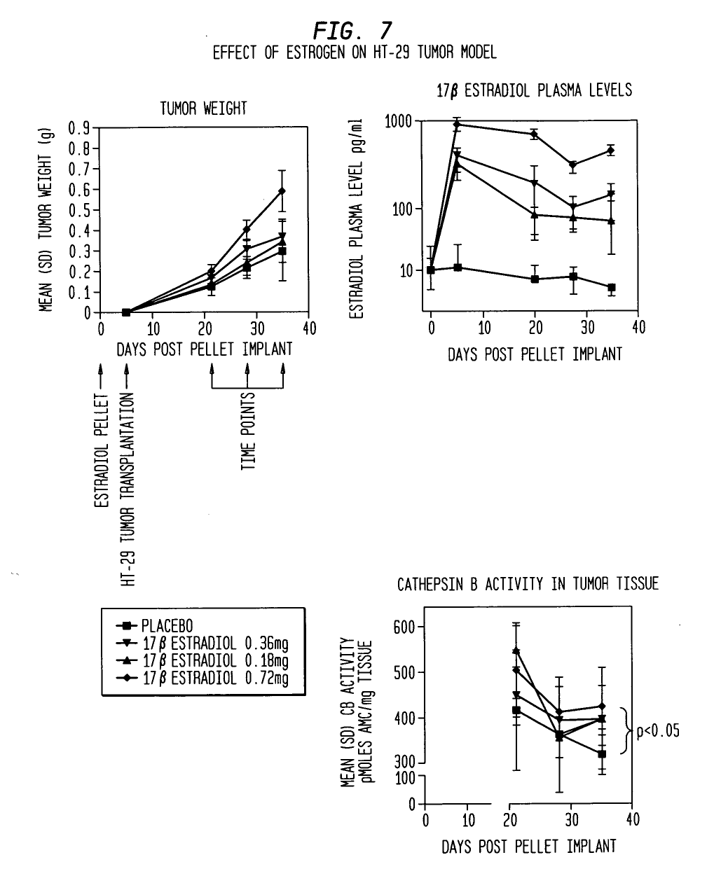

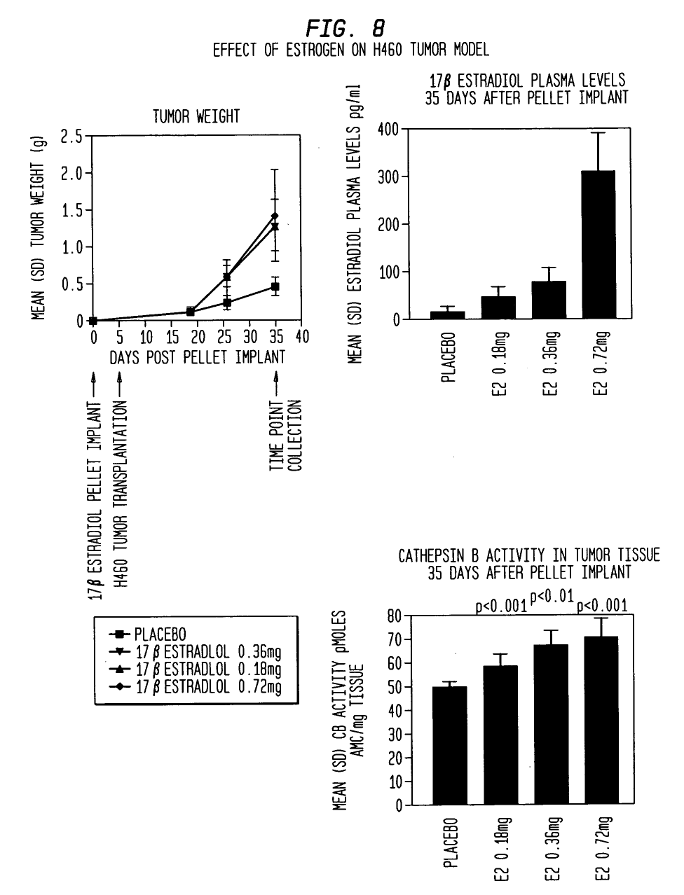

The effect of circulating estrogen levels on tumor growth and cathepsin B activity was demonstrated in the HT-29 human CRC xenograft tumor model. The results indicated that increased levels of estrogen (17β estradiol) resulted in increases in both tumor weight and cathepsin B activity (FIG. 7). A similar effect was observed in the H460 human NSCLC xenograft tumor model (FIG. 8).

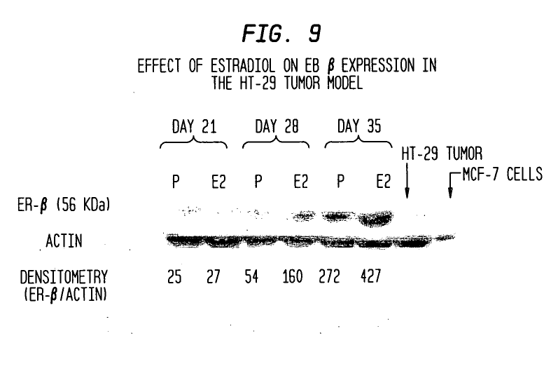

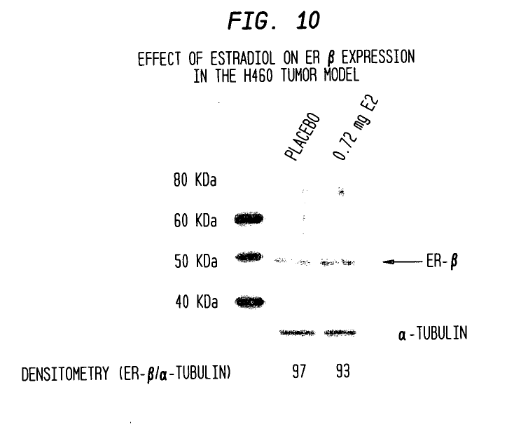

The effect of estradiol on ERβ expression in HT-29 human CRC and H460 human NSCLC was analyzed by western blot (FIGS. 9 and 10, respectively.).

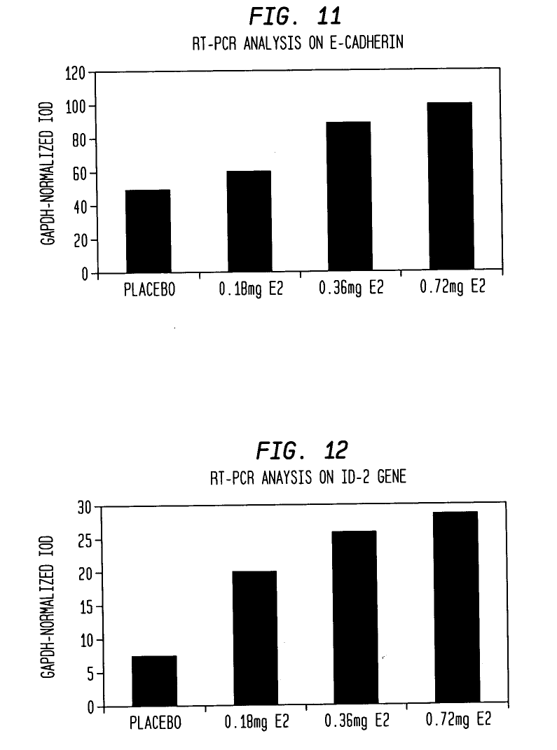

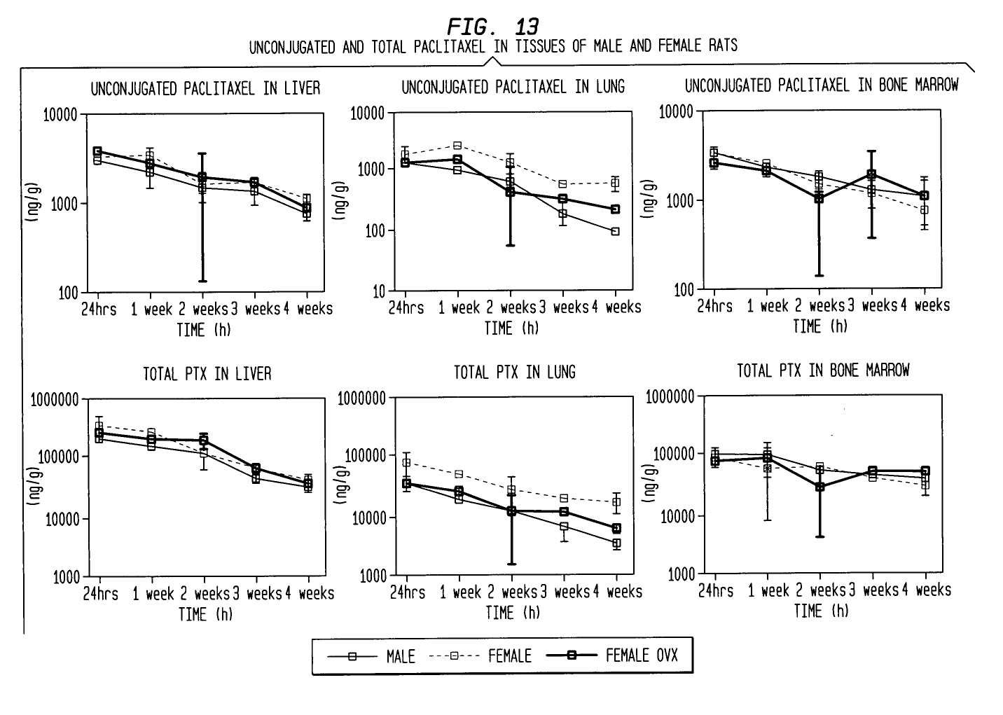

The effect of estradiol on ERβ activation in HT-29 human CRC and H460 human NSCLC cells was analyzed by RT-PCR, where increased expression of the E-Cadherin gene (in HT-29 cells) and increased expression of the Id-2 gene (in H460 cells) is indicative of ERP activation (FIGS. 11 and 12, respectively.).

The effect of estrogen treatment on PPX metabolism was demonstrated utilizing the HT-29 CRC xenograft tumor model. The results are presented in Table 4 (below). (+(E2): tumor bearing mice supplemented with 0.72 mg 17β Estradiol; −(P): tumor bearing mice not supplemented. Data from pool of 3 tumors). Higher concentrations of unconjugated paclitaxel were observed in the tumor after administration of PPX in comparison with those achieved when unconjugated paclitaxel as such was given.

TABLE 4

| | |
|---|---|
| PPX | |
| iv 90 mg/kg | |
| PTX eq. | Paclitaxel |

| Estradiol | Time | Total | Free | iv 90 mg/kg |
|---|---|---|---|---|
| treatment | (h) | PTX | PTX | PTX |
| +(E2) | 24 | 93300 | 11200 | 6810 |
| −(P) | 24 | 114700 | 17900 | 7310 |
| +(E2) | 168 | 29230 | 8480 | 421 |
| −(P) | 168 | 94300 | 28100 | nd |

Overall, the results presented in example 5 indicate that estrogen-mediated signaling is predominantly dependent on ERβ, E2 supplements induce ERβ activation and/or expression, and that increased cathepsin B activity is associated with enhanced PPX antitumor activity in HT-29 cells.

### EXAMPLE 6

The experiments and results presented in example 6 describe of free and total paclitaxel in various tissues, in the presence or absence of estrogen, utilizing a rat model. For these studies, rats were divided into three groups: 1) males, 2) sham operated (i.e., intact) females, and 3) oophorectomized females. Rats were given ¹⁴C-CT2103 (¹⁴C labeled conjugated paclitaxel, i.e., ¹⁴C labeled PPX) as a single intravenous injection of 25 mg/Kg.

At various timepoints post-injection, three rats per group were euthanized and assayed for unconjugated and total paclitaxel in various tissues (i.e., lung, liver and bone marrow).

Results from this study indicate that both unconjugated paclitaxel and total paclitaxel accumulated to statistically higher levels in the lung tissue of sham-operated female rats vs. those levels in male or oophorectomized female rats. By contrast, there was no statistical difference in drug accumulation among male, sham-operated female, or oophorectomized female rats in liver or bone marrow (FIG. 13). Collectively, these data establish that paclitaxel accumulates to statistically higher levels in the lung tissue in the presence of estrogen.

All publications cited in the specification, both patent publications and non-patent publications, are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating cancer in patients, comprising administering an anti-cancer agent comprising a poly(amino acid) polymer conjugated to an anti-cancer drug to a patient identified as having a premenopausal estrogen level and who is diagnosed with cancer, wherein the cancer is characterized by presence of estrogen receptor-bearing cancer cells, or wherein the cancer originated in a tissue or organ containing estrogen receptor bearing cells.

2. The method of claim 1, wherein the patient is identified as a female.

3. The method of claim 1, wherein the female is less than 55 years old.

4. The method of claim 1, wherein the patient is identified as a postmenopausal female on a hormone replacement therapy.

5. The method of claim 1, wherein the patient is identified as a male on a hormone replacement therapy.

6. The method of claim 1, wherein the patient is identified as a male on an anti-androgen therapy.

7. The method of claim 1, wherein the poly(amino acid) polymer is poly(glutamic acid).

8. The method of claim 1, wherein the poly(amino acid) polymer is poly(lysine).

9. The method of claim 1, wherein the anti-cancer drug is a taxane.

10. The method of claim 9, wherein the anti-cancer drug is paclitaxel.

11. The method of claim 1, wherein the anti-cancer drug is paclitaxel and the polymer comprises poly(glutamic acid).

12. The method of claim 11, wherein said conjugate is administered in an amount of about 175 to about 250 mg/m² paclitaxel equivalents.

13. The method of claim 1, wherein said conjugate is delivered intravenously.

14. The method of claim 1, wherein the cancer is lung cancer.

15. The method of claim 14, wherein the lung cancer is non-small cell lung cancer.

16. A method of treating a patient diagnosed with cancer, and identified as having a postmenopausal estrogen level, wherein the cancer is characterized by presence of estrogen receptors on the cancer cells, or wherein the cancer originates in a tissue or organ containing estrogen receptor bearing cells, and wherein said treatment comprises administering to the patient an anti-cancer agent comprising a poly(amino acid) polymer conjugated to an anti-cancer drug, and estrogen therapy.

17. The method of claim 16, wherein the patient is male.

18. The method of claim 16, wherein the patient is female.

19. The method of claim 18, wherein the female is at least 55 years old.

20. A method for selecting a cancer treatment regimen based on estradiol levels, comprising:
determining whether the serum or plasma level of estrogen of a cancer patient is premenopausal, or postmenopausal, wherein a premenopausal estrogen level indicates that treatment with paclitaxel conjugated to polyglutamate will be more effective than unconjugated paclitaxel, and wherein a low postmenopausal estrogen level in serum or plasma indicates that treatment with paclitaxel conjugated to polyglutamate will be no more effective than unconjugated paclitaxel.
